(19) United States
(12) Patent Application Publication
(10) Pub. No.: US 2019/0083368 A1
(43) Pub. Date: Mar. 21, 2019

(54) PIEZOELECTRIC COSMETIC COMPOSITION

(71) Applicant: COSMAX, INC., Gyeonggi-do (KR)

(72) Inventors: Sung Yun HONG, Gyeonggi-do (KR); Ji Hui JANG, Gyeonggi-do (KR); Su Ji KIM, Gyeonggi-do (KR); Jun Bae LEE, Gyeonggi-do (KR); Youn Joon KIM, Seoul (KR); Kweon Jong YOO, Gyeonggi-do (KR); Hui Yun HWANG, Gyeongsangbuk-do (KR); Jun Hyeok AHN, Daegu (KR); Sang Gyun HWANG, Gyeongsangbuk-do (KR)

(73) Assignee: COSMAX, INC., Gyeonggi-do (KR)

(21) Appl. No.: 16/082,421

(22) PCT Filed: Jan. 23, 2017
(86) PCT No.: PCT/KR2017/000770

§ 371 Date: Sep. 5, 2018

(30) Foreign Application Priority Data
Aug. 26, 2016 (KR) 10-2016-0109329

(51) Int. Cl. A61K 8/02; A61K 8/27; A61K 8/29; A61K 8/34; A61K 8/86; A61K 8/06; A61Q 19/00

(52) U.S. Cl. CPC A61K 8/0295; A61K 8/27; A61K 8/29; A61K 8/342; A61K 2800/805; A61K 8/86; A61K 8/06; A61Q 19/00; A61K 8/345

(57) ABSTRACT

The present disclosure relates to a cosmetic composition having piezoelectricity. More particularly, the present disclosure relates to a piezoelectric cosmetic composition which includes a piezoelectric material capable of generating microcurrent upon the application of pressure as an active ingredient.

According to the present disclosure, when the piezoelectric composition is applied to and massaged on the skin, it is possible to maximize an effect of skin care, including an effect of improving skin elasticity, by generating microcurrent.

### TECHNICAL FIELD

The present disclosure is made with a subject number of S2341055 under the support of Korean Ministry of SMEs and Startups, and the supervisory management organ of the subject is Korea Institute for Advancement of Technology (KIAT), the subject business title is “World Class 300 R&D”, the subject title is “Discovery of Advanced Material Having Function of Controlling Skin Cell Receptor for K-Cosmetic Pacesetting Functional Cosmetics and Development of Optimized Skin Delivery Technique Containing The Same”, the supervisor organ is COSMAX, and the research period is Sep. 1, 2013 to Aug. 31, 2017.

The present disclosure relates to a cosmetic composition having piezoelectricity. More particularly, the present disclosure relates to development of a piezoelectric cosmetic composition which generates microcurrent upon the application of pressure to enhance skin elasticity.

### BACKGROUND

In the human body, microcurrent flows in order to transmit signals between organs. In general, when one's health is in decline or one's physical condition becomes unstable, microcurrent flowing in the body becomes weak. To supplement this, an instrument generating microcurrent is used so that microcurrent may flow in the body to enhance activity. According to [Military Medical Research, 2014, 1:24], microcurrent stimulates wounded skin cells, and thus helps healing. According to [Journal of Physical Therapy Science, 2011, 23, 515-518], it is known that microcurrent increases the blood flow rate, and thus helps blood circulation. Particularly, according to [Homeopathy, 2012, 101, 147-153], it is known that when microcurrent is introduced to the skin, collagen fibers become thick and skin density is increased to provide an effect of enhancing skin elasticity. According to [Clin Orthop, 1982, 171, 264-272], it is known that introduction of microcurrent enhances absorption of amino acids.

Recently, based on the above-mentioned results, cosmetic instruments for generating microcurrent have been commercialized. Particular examples thereof include Korean Patent Publication No. 10-1050069 (Portable Massage Instrument Having Function of Introducing Ions), Korean Patent Publication No. 10-0614799 (Portable Skin Cosmetic Instrument), and Korean Patent Publication No. 10-1209102 (Microcurrent Instrument Having Electrostimulation Function). In addition, skin control has been made frequently by a method of introducing an active ingredient, which has a difficulty in reaching the derma, with the aid of a skin specialist. However, there have been problems related with charging or battery mounting during use of a cosmetic instrument, failure in the instrument, poor portability, low cost efficiency, troubles in visiting dermatologists, irritation caused by injection, or the like.

Therefore, the inventors of the present disclosure have conducted many studies about generation of microcurrent easily without using a separate cosmetic instrument in order to overcome the above-mentioned limitations caused by the cosmetic instruments or the aid of skin specialists. We have found that when a piezoelectric material capable of generating microcurrent upon the application of pressure is incorporated to a cosmetic composition, it is possible to generate microcurrent naturally when the cosmetic composition is applied to the skin and is pushed or rubbed on the skin, and thus to help skin care. The present disclosure is based on this finding.

### SUMMARY

### Technical Problem

The present disclosure is designed to solve the problems of the related art, and therefore the present disclosure is directed to providing a piezoelectric cosmetic composition which can maximize a skin massage effect and skin care effect by incorporating a piezoelectric material to a cosmetic composition and inducing microcurrent upon the skin application in order to avoid the disadvantages occurring when using a cosmetic instrument for generating microcurrent.

### Technical Solution

In one general aspect, there is provided a piezoelectric cosmetic composition applied to the skin, which includes a piezoelectric material capable of generating microcurrent upon the application of pressure as an active ingredient.

A piezoelectric phenomenon includes generation of electric charges (electric energy) through a change in internal polarization by deformation (mechanical energy) when applying pressure to a material. Typical piezoelectric materials include inorganic piezoelectric elements, such as barium titanate (BaTiO₃), lead zirconate titanate (Pb(Zr,Ti)O₃, PZT). Recently, PVDF has been given many attentions as a piezoelectric polymer material. In addition, solid materials, such as metal oxides, are used. Such materials have been used in various industrial fields, including sensors, filters, resonators, actuators, speakers, energy harvesting, or the like. However, they have never been used in the field of cosmetic compositions.

According to the present disclosure, the piezoelectric material can generate microcurrent when pressure is applied thereto. Thus, when a cosmetic agent including the piezoelectric material is applied to the skin and pushed and rubbed thereon, the piezoelectric material can generate microcurrent and thus helps skin care. According to the present disclosure, the piezoelectric material preferably shows a piezoelectric constant of 1-20 pC/N as determined by using a liquid phase piezoelectricity measuring device. After the inventors of the present disclosure designed a device for determining the piezoelectricity of a liquid material (see, Korean Patent Application No. 10-2016-95633) and cosmetic compositions including various types of piezoelectric materials were determined for piezoelectricity, it was shown that the piezoelectric materials showed a piezoelectric constant within the above-defined range.

According to the present disclosure, the piezoelectric material may include any known piezoelectric material capable of generating microcurrent upon the application of pressure. Preferably, the piezoelectric cosmetic composition according to the present disclosure includes a liquid crystal emulsion itself, inorganic pigment powder, piezoelectric element or a piezoelectric polymer.

The inventors of the present disclosure got an idea from a liquid crystal phase with piezoelectricity used in a different type of industry, i.e., liquid crystal display industry, and focused about a liquid crystal emulsion used to date as a cosmetic agent and also having piezoelectricity. Then, a liquid crystal emulsion and conventional emulsion were prepared and determined for piezoelectricity. As a result, it was found that a liquid crystal emulsion, unlike a conventional emulsion, shows piezoelectricity, thereby enhancing skin elasticity.

According to the present disclosure, the liquid crystal emulsion used as a piezoelectric material may be any liquid crystal emulsion prepared by a liquid crystal emulsification process known to date in the field of cosmetics. For example, the liquid crystal emulsion may be a thermotropic liquid crystal which undergoes a change depending on temperature or a lyotropic liquid crystal which undergoes a change depending on compositional variations. Particularly, the lyotropic liquid crystal may be obtained by using an aliphatic alcohol, such as cetyl alcohol or stearyl alcohol, cholesterol, cholesteryl miristate or ceramide, or a phospholid, such as lecithin.

According to the present disclosure, the inorganic pigment powder used as a piezoelectric material may be any inorganic pigment powder used to date in the field of cosmetics. Particular examples of the inorganic pigment powder may include not only a white pigment, such as titanium dioxide (TiO₂) or zinc oxide (ZnO) but also an extender pigment, such as silica (SiO₂) or mica. After a liquid crystal emulsion or conventional emulsion was combined with various types of inorganic pigment powder and determined for piezoelectricity, it has been found that the liquid crystal emulsion showed piezoelectricity, unlike the conventional emulsion, and piezoelectricity was increased as the amount of the inorganic pigment was increased.

According to the present disclosure, the piezoelectric element used as a piezoelectric material may include a piezoelectric material known to date as a piezoelectric element, such as crystal, barium titanate, lead titanate zirconate or piezoelectric ceramics obtained by sintering them. In addition, the piezoelectric polymer used as a piezoelectric material may include known piezoelectric polymers, such as polyvinylidene fluoride (PVDF), polyamides or ParyleneC.

According to the present disclosure, the piezoelectric material may be a part or the whole of a cosmetic composition, when it is a liquid crystal emulsion itself. In addition, when the piezoelectric material is inorganic pigment, or the like, it is present in an amount of 1-10 wt % based on the total weight of the piezoelectric cosmetic composition.

In the piezoelectric cosmetic composition according to the present disclosure, the microcurrent generated by the piezoelectric material not only helps infiltration of a cosmetic agent into the skin but also improves skin elasticity through skin stimulation. The inventors of the present disclosure have found that the cosmetic composition including the piezoelectric material enhances skin elasticity, as compared to a cosmetic composition not including the piezoelectric material, and thus helps skin activity.

According to the present disclosure, the piezoelectric cosmetic composition may include ingredients used generally in a cosmetic composition, except the piezoelectric material used as an active ingredient. Such ingredients may include at least one cosmetic agent selected from the group consisting of water, oil, polyol, surfactant and a moisturizing agent.

According to the present disclosure, the cosmetic composition has a formulation selected from an emollient, skin softener, lotion, cream and essence.

### Advantageous Effects

According to the embodiments of the present disclosure, it is possible to maximize an effect of skin care, including an effect of improving skin elasticity, by incorporating a piezoelectric material capable of generating microcurrent to a cosmetic composition so that microcurrent may be generated when the cosmetic composition is applied to and massaged on the skin.

### BRIEF DESCRIPTION OF THE DRAWINGS

FIG. 1 is a schematic view illustrating an apparatus for determining the piezoelectricity of a cosmetic agent. A liquid specimen is introduced to a sealed container and quantity of charge is measured between electrodes positioned at both sides of the specimen while applying a vertical load thereto.

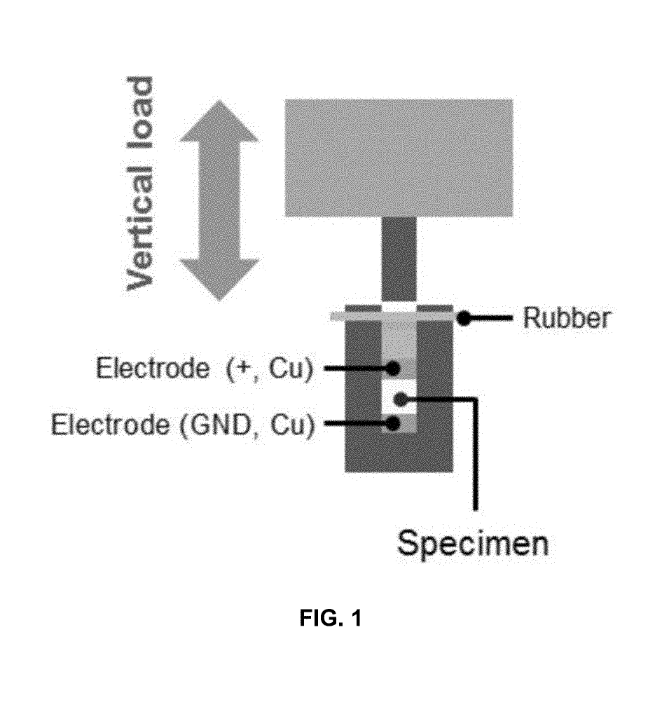

FIG. 2 is a graph illustrating the piezoelectricity of the cosmetic compositions according to Comparative Examples 1 and 2 and Examples 1-3.

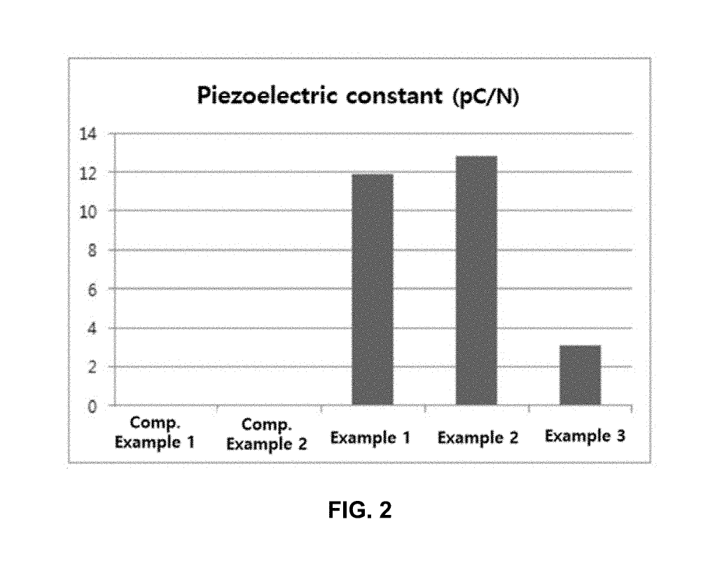

### DETAILED DESCRIPTION

Exemplary embodiments now will be described more fully hereinafter. The present disclosure may, however, be embodied in many different forms and should not be construed as limited to the exemplary embodiments set forth therein.

Test Example 1: Preparation of Comparative Examples and Examples

To carry out comparison of the effect of a cosmetic composition in the presence or absence of a piezoelectric material as an active ingredient according to the present disclosure, emulsion was prepared with the composition as shown in the following Table 1.

Each of Comparative Examples 1 and 2 is a conventional (W/O) emulsion, Example 1 is a liquid crystal emulsion, Example 2 is a conventional emulsion containing zinc oxide as a piezoelectric material, and Example 3 is a conventional emulsion containing titanium dioxide as a piezoelectric material.

TABLE 1

| | Comparative | Comparative | | | |
|---|---|---|---|---|---|
| Ingredients | Example 1 | Example 2 | Example 1 | Example 2 | Example 3 |
| 1. polydecene | 0.009 | — | — | — | — |
| 2. Squalane | 0.0005 | — | — | — | — |
| 3. Cetearyl alcohol | — | — | 4 | — | — |
| 4. C14-22 alcohol | — | — | 1.6 | — | — |
| 5. Vitamin E acetate | — | 0.2 | — | 0.2 | 0.2 |
| 6. Glyceryl stearate SE | — | — | 0.5 | — | — |
| 7. Caprylic/capric triglyceride | — | 3 | 10 | 3 | 3 |
| 8. PEG/PPG-19/19 dimethicone | — | 1.5 | 0.1 | 1.5 | 1.5 |
| 9. Dimethicone | 3 | — | — | — | — |
| 10. Cyclomethicone | 3 | — | — | — | — |
| 11. Cetyl dimethicone | 3 | — | — | — | — |
| 12. Dimethicone/vinyl dimethicone | 3 | — | — | — | — |
| crosspolymer | | | | | |
| 13. cyclopentasiloxane | — | 21.5 | — | 21.5 | 21.5 |
| 14. C12-20 alkyl glucoside | — | — | 0.4 | — | — |
| 15. Hydrogenated lecithin | — | — | 0.1 | — | — |
| 16. D.I. Water | To 100 | To 100 | To 100 | To 100 | To 100 |
| 17. 1% EDTA-2Na solution | 2 | 2 | 2 | 2 | 2 |
| 18. Sodium PCA | 0.5 | — | — | — | — |
| 19. Sodium glutamate | — | 0.5 | — | 0.5 | 0.5 |
| 20. Dipropylene glycol | — | 10 | — | 10 | 10 |
| 21. Butylene glycol | 2 | — | — | — | — |
| 22. Glycerin | 5 | 10 | 15 | 10 | 10 |
| 23. 1% Carbomer solution | — | — | 12 | — | — |
| 24. Triethanolamine | — | — | 0.1 | — | — |
| 25. presevatives | suitable | suitable | suitable | suitable | suitable |
| | amount | amount | amount | amount | amount |
| 26. Perfume | 0.1 | 0.1 | 0.1 | 0.1 | 0.1 |
| 27. piezoelectric material | — | — | — | 4 | — |
| (zinc oxide) | | | | | |
| 28. piezoelectric material | — | — | — | — | 4 |
| (titanium dioxide) | | | | | |

1) In Table 1, Ingredients 1-15 are oil-soluble ingredients, wherein Ingredients 3 and 4 are higher aliphatic alcohols, Ingredients 14 and 15 are non-polyethylene oxide (POE) based nonionic surfactants, and Ingredients 16-24 are water-soluble ingredients, wherein Ingredients 20-22 are polyols, Ingredients 23 and 24 are viscosity modifiers, Ingredient 25 is a preservative, Ingredient 26 is a fragrance ingredient, and Ingredients 27 and 28 are piezoelectric materials.

2) In Table 1, Ingredients 1-15 were mixed and dissolved at 70-80° C. to form an oil phase solution.

3) In a separate container, Ingredients 16-22 described in Table 1 were mixed and warmed/dissolved to a temperature of 70-80° C. to form an aqueous phase solution.

4)-1. The oil phase solution of 2) was added gradually to the aqueous phase solution and agitated with a homomixer (Example 1).

4)-2. The aqueous phase solution of 3) was added gradually to the oil phase solution of 2) and agitated with a homomixer (Comparative Examples 1 and 2, and Examples 2 and 3).

5) Ingredient 23 in Table 1 was mixed with the resultant mixed solution of 4), and then Ingredient 23 was added thereto.

6) Example 1 was quenched to 40° C. within 10 minutes after the completion of the process of 5). Then, Ingredients 25 and 26 in Table 1 were introduced, followed by degassing.

7) In the case of Comparative Examples 1 and 2, the mixture was allowed to stand at room temperature after the completion of the process of 5), and Ingredients 25 and 26 in Table 1 were introduced, followed by degassing.

8) In the case of Examples 2 and 3, the mixture was allowed to stand at room temperature after the completion of the process of 5), Ingredients 25 and 26 in Table 1 were introduced, and each of the piezoelectric materials 27 and 28 was dispersed, followed by degassing.

The liquid crystal emulsion according to Example 1 were obtained by mixing non-POE based nonionic lecithin with a glucoside-based surfactant. It is a liquid crystal emulsion having a stable multilayer lamella structure containing intercellular lipid after the quenching process. After the liquid crystal emulsion was observed with a polarizing microscope, it was shown that a liquid crystal phase was formed.

Test Example 2: Piezoelectricity Test for Piezoelectric Cosmetic Compositions

Examples 1-3 and Comparative Example 1 and 2 obtained according to Test Example 1 were determined for piezoelectricity by using a liquid phase piezoelectricity measuring apparatus. The results are shown in the following Table 2.

<Method>

1) First, 55 μL of each sample was inserted between electrodes and a motor stage was moved to apply a load of 4-10N repeatedly.

2) Quantity of charge generated from the applied load and sample was measured.

TABLE 2

| | Comparative | Comparative | Exam- | Exam- | Exam- |
|---|---|---|---|---|---|
| Test | Example 1 | Example 2 | ple 1 | ple 2 | ple 3 |
| piezoelectric | — | — | 11.9 | 12.8 | 3.1 |
| constant | | | | | |
| (pC/N) | | | | | |

As shown in Table 2, Comparative Examples 1 and 2 (conventional W/O emulsion) showed no piezoelectricity, while Example 1 (liquid crystal emulsion) had a piezoelectric constant value of 11.9 corresponding to a level of a polymer piezoelectric element. Examples 2 and 3 (piezoelectric material-containing emulsion) had a piezoelectric constant of 12.8 and 3.1, respectively. Therefore, the highest piezoelectricity was observed from the emulsion containing zinc oxide.

Test Example 3: Skin Safety Test for Piezoelectric Cosmetic Compositions

Ten male and female adult subjects suffering from no skin diseases were allowed to use Examples 1-3 and Comparative Examples 1 and 2, and to evaluate a degree of skin irritation as described hereinafter. First, 20 μL of a specimen was applied to the forearm of each subject, and the test site was sealed and covered with a patch for 24 hours. Then, 30 minutes and 24 hours after removing the patch, the skin response was examined according to the terminology defined in the guideline of CTFA (Cosmetic, Toiletry and Fragrance Association). The results were obtained by averaging the points judged by the subjects and evaluated as follows: 0 (no irritation), less than 1 (low irritation), less than 3 (light irritation), less than 5 (moderate irritation), 5 or higher (strong irritation). The irritation results are shown in the following Table 3.

TABLE 3

| | Comparative | Comparative | Exam- | Exam- | Exam- |
|---|---|---|---|---|---|
| Test | Example 1 | Example 2 | ple 1 | ple 2 | ple 3 |
| Skin Irritation | 0 (no | 0 (no | 0 (no | 0 (no | 0 (no |
| Index (PII) | irri- | irri- | irri- | irri- | irri- |
| | tation) | tation) | tation) | tation) | tation) |

As shown in Table 3, Examples 1-3 and Comparative Examples 1 and 2 cause no irritation. Thus, it was shown that they can be used safely for cosmetics.

Test Example 4: Evaluation of Skin Elasticity for Piezoelectric Cosmetic Compositions

Comparative Example 2 (conventional W/O emulsion) and Example 1 (liquid crystal emulsion) according to Test Example 1 were evaluated for skin elasticity. Each of Comparative Example 2 and Example 1 was applied to five males and females in a predetermined amount. Two weeks and four weeks after the application, skin elasticity was determined. The results are shown in the following Table 4.

TABLE 4

| Skin | Comparative | |
|---|---|---|
| Elasticity | Example 2 | Example 1 |
| 2 weeks | 3.7 | 4.4 |
| 4 weeks | 5.3 | 8.8 |

After determining skin elasticity, it was shown that skin elasticity was increased by 1.4 times after 4 weeks from 3.7 (2 weeks) in the case of Comparative Example 2. On the contrary, in the case of Example 1 (liquid crystal emulsion), skin elasticity was increased by 2 times after 4 weeks as compared to the skin elasticity after 2 weeks.

As can be seen from the foregoing, the piezoelectric cosmetic composition according to the present disclosure may be used as a cosmetic agent that helps skin activity.

### REFERENCES

(Patent Document 1) KR10-1050069 B

(Patent Document 2) KR10-0614799 B

(Patent Document 3) KR10-1209102 B

What is claimed is:

1. A piezoelectric cosmetic composition applied to the skin, which comprises a piezoelectric material capable of generating microcurrent upon the application of pressure as an active ingredient.

2. The piezoelectric cosmetic composition according to claim 1, wherein the piezoelectric material has a piezoelectric constant of 1-20 pC/N as determined by using a liquid phase piezoelectricity measuring device.

3. The piezoelectric cosmetic composition according to claim 1, wherein the piezoelectric material is a liquid crystal emulsion itself, inorganic pigment powder, piezoelectric element or a piezoelectric polymer.

4. The piezoelectric cosmetic composition according to claim 1, wherein the microcurrent generated by the piezoelectric material not only helps infiltration of a cosmetic agent into the skin but also improves skin elasticity through skin stimulation.

5. The piezoelectric cosmetic composition according to claim 1, which further comprises at least one cosmetic agent selected from the group consisting of water, oil, polyol, surfactant and a moisturizing agent.

6. The piezoelectric cosmetic composition according to claim 1, which has a formulation selected from an emollient, skin softener, lotion, cream and essence.
